(19) United States
(12) Patent Application Publication
(10) Pub. No.: US 2019/0282621 A1
(43) Pub. Date: Sep. 19, 2019

(54) BLOOD DERIVATIVES COMPOSITE MATERIAL, METHODS OF PRODUCTION AND USES THEREOF

(71) Applicant: ASSOCIATION FOR THE ADVANCEMENT OF TISSUE ENGINEERING AND CELL BASED TECHNOLOGIES & THERAPIES A4TEC-, Braga (PT)

(72) Inventors: Bárbara Bruna DA SILVA MENDES, Fátima (PT); Rui Miguel DE ANDRADE DOMINGUES, Guimarães (PT); Maria Manuela ESTIMA GOMES, Porto (PT); Pedro Miguel DE SOUSA BABO, Viana do Castelo (PT); Rui Luís GONÇALVES DOS REIS, Porto (PT)

(73) Assignee: ASSOCIATION FOR THE ADVANCEMENT OF TISSUE ENGINEERING AND CELL BASED TECHNOLOGIES & THERAPIES A4TEC-, Braga (PT)

(21) Appl. No.: 16/345,088

(22) PCT Filed: Oct. 27, 2017
(86) PCT No.: PCT/IB2017/056699

§ 371 Date: Apr. 25, 2019

(30) Foreign Application Priority Data
Oct. 27, 2016 (PT) 109701

(51) Int. Cl. A61K 35/19; A61K 9/51; A61K 9/00

(52) U.S. Cl. CPC A61K 35/19; A61K 9/0019; A61K 9/51

(57) ABSTRACT

The present disclosure relates to a blood derivatives based nanocomposite materials incorporating comprising oxidized cellulose nanocrystals, methods for their production, and uses thereof. Also disclosed herein is a method for the production of oxidized cellulose nanocrystals with gradients of sulfation degree and their use to modulate the affinity of protein content of blood derivatives/cellulose nanocrystals nanocomposite materials. Therefore, the present disclosure is useful use in regenerative medicine and/or tissue engineering.

### TECHNICAL FIELD

The present disclosure relates to a blood derivatives nanocomposite material comprising oxidized cellulose nanocrystals, methods for their production, and uses thereof. Also disclosed herein is a method for the production of oxidized cellulose nanocrystals with gradients of sulfation degree and their use to modulate the affinity of protein content of blood derivatives/cellulose nanocrystals nanocomposite materials.

### BACKGROUND

Blood is composed of different cellular, sub-cellular and molecular components that are involved in essential stages of wound healing and regenerative processes.

The separation of blood components results on different blood derivative (BD) formulations.

BD have shown promising features as an autologous and natural reservoir of supra-physiological doses of growth factors (GFs), cytokines, and extracellular matrix (ECM) precursors which are known to significantly modulate cell behaviour.

Among the ECM precursors present in BD are fibrinogen and fibronectin.

Fibrinogen of BD has been commonly activated by calcium, collagen and exo- or/and endogenous thrombin, which promote the polymerization of fibrinogen producing a stable fibrin matrix.

The use of different types of BD has shown positive clinical effects in several fields of regenerative medicine such as in the treatments of tendon injures and pathologies, cartilage disorders, as well as in bone, periodontal and soft tissue wound healing.

BD have been incorporated within polymeric matrices or used as biomaterials by self-crosslinking of its protein content in order to improve or tune the biological response of these biomaterials.

Traditional methods of BD application in tissue engineering (TE) strategies rely on the activation of BD by thrombin and calcium activation in order to form a clot.

Currently, BD-based strategies have several limitations, including: lack of standardization, limited mechanical properties, fast degradation of the biological active substances, limited in vitro/in vivo stability, without sufficient control over bioactive molecules release and low retention at the injury site.

There is a need to overcome BD-based strategies namely, shrinkage upon cellular encapsulation, modulation in the temporal and spatial demands of growth factor release, control over scaffold degradation rate and improved native tissue integration.

The development of more controllable systems for the delivery of well characterized populations of biomolecules will certainly improve the clinical outcomes of the use of BD.

In native ECM microenvironments, GFs are protected and stabilized via their binding to different ECM components that regulate their availability and signalling. In a biomimetic strategy, researchers have combined BD with different biomaterials to modulate the delivery of bioactive molecules in order to guide the wound healing process.

Cellulose nanocrystals (CNC) present outstanding characteristics namely high biocompatibility, low density, high surface area, high mechanical properties and a reactive surface which enables different surface chemical modifications. The nanodimensions and superior strength of CNC make it an ideal reinforcing material to a low strength matrix.

Biomaterials containing surface sulfated CNC can exhibit specific or unspecific interactions with the pool of GFs released from PL. These interactions of platelet-released GFs and other proteins with the sulfated CNC within the biomaterial matrix may increase their local concentration/specificity within the 3D microenvironment, thus enhancing/tuning their effect over encapsulated stem cells. However, the use of CNC with a gradient of surface SO³⁻ half-ester groups as mimicry of ECM sulfated GAGs, has not been previously proposed.

WO2014077854 A1 discloses a system and method for the production of a fibrin matrix that incorporates CNC and/or oxidized CNC.

WO2013116791 A1 discloses the use of biomaterials in combination of blood products.

These facts are disclosed in order to illustrate the technical problem addressed by the present disclosure.

### GENERAL DESCRIPTION

The present disclosure disclose use of CNC as a biomaterial or precursor in combination with BD component.

The present disclosure also described the use of BD as an intermediate component of the process to produce biomaterials, namely sponges and hydrogels, to be used as scaffolds or cell carries in TE applications.

Biomaterials containing surface sulfated CNC can exhibit specific or unspecific interactions with the pool of GFs released from PL. These interactions of platelet-released GFs and other proteins with the sulfated CNC within the biomaterial matrix may increase their local concentration/specificity within the 3D microenvironment, thus enhancing/tuning their effect over encapsulated stem cells. It was surprisingly observed, the use of CNC with a gradient of surface SO³⁻ half-ester groups as mimicry of ECM sulfated GAGs.

In the present disclosure, a BD component is any therapeutic substance prepared from human blood. This includes: whole blood; blood components; and plasma derivative. In particular platelet, platelet released content, platelet-rich plasma, or their combinations.

An aspect of the present disclosure relates to a composition comprising:
a BD component;
a CNC as a filler comprising carbonyls at the CNC surface;
wherein such CNC comprises a sulfation degree of at least 50 mmolKg⁻¹; between 80 and 500 mmolKg⁻¹; more preferably between 100 and 300 mmolKg⁻¹; even more preferably between 120 and 300 mmolKg⁻¹.

It was surprisingly found that certain sulfation degree promotes favoured non-covalently interactions with the protein content derived from BD formulations. The sulfation degree of the CNC may be measured by the method conductometric titration.

In an embodiment for better results, the composition may comprise 0.05-2% w/v of CNC; preferably 0.1-1% w/v; more preferably 0.15-0.61% w/v.

In an embodiment for better results, the composition may comprise 0.5×10⁴ platelets/μL-1×10⁸ platelets/μL of BD platelet concentration; preferably 1×10⁵-1×10⁷ platelets/μL; more preferably 1×10⁶-5×10⁶ platelets/μL.

In an embodiment for better results, the composition may comprise thrombin, calcium, calcium salts, or mixtures thereof. It was surprisingly found that thrombin and calcium may be use to the activation of the coagulation cascade to convert fibrinogen contained in the BD into fibrin.

In an embodiment for better results, the composition may comprise 0.1 U.mL⁻¹-50 U.mL⁻¹ of thrombin preferably 0.5 U.mL⁻¹-5 U.mL⁻¹; more preferably 1 U.mL⁻¹-3 U.mL⁻¹.

In an embodiment for better results, the composition may comprise 0.1 mM-25 mM of calcium or calcium derivate; preferably 0.5 mM-10 mM; more preferably 1 mM-5 mM.

In an embodiment for better results, the composition may comprise an amount of carbonyl groups at the surface of the CNC between 0.01-8 mmol.g⁻¹; preferably 0.1-4 mmol.g⁻¹; more preferably 0.4-0.9 mmol.g⁻¹.

In an embodiment for better results, the composition may further comprise one or more active ingredient or biomolecule.

In an embodiment for better results, the composition may comprise as an active ingredient or biomolecule: active ingredient or biomolecule is: a drug; an active ingredient, a growth hormone, a cell attractant, a drug molecule, a cell, a bioactive glass, a tissue growth promoter, a cell attractant, or combinations thereof.

In an embodiment for better results, the drug molecule may be an anti-inflammatory, antipyretic, analgesic, anticancer agent, or mixtures thereof.

In an embodiment for better results, the wherein cells may be selected from: osteoblasts, osteoclasts, osteocytes, pericytes, endothelial cells, endothelial progenitor cells, bone progenitor cells, hematopoietic progenitor cells, hematopoietic stem cells, neural progenitor cells, neural stem cells, mesenchymal stromal/stem cells, induced pluripotent stem cells, embryonic stem cells, or combinations thereof.

In an embodiment for better results, the composition may further comprise one or more pharmaceutically acceptable excipient. In particular an additive, a binder, a disintegrant, a diluent, a lubricant, a plasticizer, or mixtures thereof.

In an embodiment for better results, the average length of the CNC is between 40-2500 nm; preferably 100-500 nm; more preferably 200-300 nm.

In an embodiment for better results, average width of the CNC is between 2-50 nm; preferably 3-20 nm; more preferably 4-15 nm.

In an embodiment for better results, the BD component may be a fraction of blood including red blood cells, white blood cells, buffy coat, plasma or platelet rich plasma, or an extract of blood including growth factors or extracellular matrix proteins purified or released from blood, blood fractions, or combination thereof. More preferably a platelet, platelet released content, platelet-rich plasma, or combinations thereof.

In an embodiment for better results, the BD component may be obtainable by centrifugation, by apheresis, or combinations thereof.

Another aspect of the present disclosure relates to the use of the composition of the present disclosure in medicine, veterinary or cosmetic, namely for use in tissue engineering, tissue regeneration or regenerative medicine, or in cellular therapy.

In an embodiment for better results, the composition may be use the treatment or therapy of wound healing or a tissue injury defect. In particular, the treatment or therapy of defects of skin wound, orthopedic injury, pain, nerve disease, dental injury, bone injury; or diabetic wound healing.

In an embodiment for better results, the composition may be use as an injectable formulation.

In an embodiment for better results, the composition is an injectable formulation, in particular an in situ injection.

Another aspect of the present disclosure relates to a hydrogel comprising the composition of the present subject-matter and comprising a BD component reinforced with modified CNC (or oxidized CNC) of a certain sulfation degree and, thrombin and/or calcium addition.

In an embodiment for better results, hydrogels with higher CNC content showed lower degradation rate. In fact, CNC incorporation lead to an improvement of PL stability (FIG. 6).

In an embodiment for better results, the hydrogel may be an in situ crosslinked injectable hydrogels at physiological conditions.

Another aspect of the present disclosure relates to a sponge or scaffold comprising the composition described in the present disclosure comprising a BD and modified CNC of a certain sulfation degree.

Another aspect of the present disclosure relates to a sponge or scaffold comprising the composition described in the present disclosure comprising a BD and modified CNC of a certain sulfation degree, and, thrombin and/or calcium addition.

In an embodiment for better results, sponge or scaffold may be casted to the desired mold shape.

In an embodiment for better results, the sponge or scaffold may further comprise encapsulated cell and/or cells.

In an embodiment for better results, the cell or cells may be encapsulated or seeded.

Another aspect of the present disclosure, relates to a method for producing scaffolds including injectable hydrogels, hydrogels and sponges for regeneration of biological tissues based on the use of BD and modified cellulose based-biomaterial.

In an embodiment for better results, the production method may further comprise oxidation of sulfated CNC by sodium periodate reaction or 2,2,6,6-tetramethylpiperidine-1-oxyl radical.

In an embodiment for better results, the production method may further comprise a hydrothermal treatment to produce CNC with a gradient of sulfation degrees.

In an embodiment for better results, a method for producing sponge biomaterials for any tissue engineering application includes mixing BD with an aqueous suspension of oxidized CNC of a certain sulfation degree, incubating the mixture for a certain period of time, freezing and freeze-drying the crosslinked nanocomposite material.

In an embodiment for better results, the method may comprise an aqueous oxidized CNC solution of a certain sulfation degree covalently crosslinks with the amine groups of the protein content released from BD formulations.

In an embodiment for better results, the method may comprise an aqueous oxidized CNC solution of a certain sulfation degree interacts electrostatically with the positive groups of the protein content released from BD formulations.

In an embodiment for better results, a method for producing sponge biomaterial for any TE application includes mixing BD with an aqueous suspension of oxidized CNC of a certain sulfation degree, thrombin, and calcium, incubating the mixture for a certain period of time, freezing and freeze-drying the crosslinked nanocomposite material.

In an embodiment for better results, wherein oxidized CNC of a certain sulfation degree covalently crosslinks with the amine groups of the protein content derived from BD formulations.

In an embodiment for better results, wherein oxidized CNC of a certain sulfation degree interacts electrostatically with the positive groups of the protein content derived from BD formulations.

In an embodiment for better results, wherein thrombin and calcium activation of the coagulation cascade is used to convert fibrinogen contained in the BD into fibrin.

In an embodiment for better results, a method for producing hydrogels for any TE application includes mixing BD with an aqueous suspension of oxidized CNC of a certain sulfation degree, thrombin and calcium, incubating the mixture for a certain period of time, producing in situ crosslinked hydrogels.

In an embodiment for better results, wherein oxidized CNC of a certain sulfation degree covalently crosslinks with the amine groups of the protein content derived from BD formulations.

In an embodiment for better results, wherein oxidized CNC of a certain sulfation degree interacts electrostatically with the positive groups of the protein content derived from BD formulations.

In an embodiment for better results, wherein thrombin and calcium activation of the coagulation cascade is used to convert fibrinogen contained in the BD into fibrin.

### BRIEF DESCRIPTION OF THE DRAWINGS

The following figures provide preferred embodiments for illustrating the description and should not be seen as limiting the scope of invention.

FIG. 1: Decrease of sulfate group content of oxidized CNC by thermal degradation.

FIG. 2: Schematic representation of the affinity between CNC surface and PL-derived proteins.

FIG. 3: Schematic representation of the preparation of PL enriched CNC injectable hydrogels. A) PL and B) oxidized CNC and preparation of PL-CNC hydrogel.

FIG. 4: Freeze dried spongy hydrogels (A) before and (B) after immersion in PBS with varying CNC content wherein the CNC concentration is 0% w/v (PL-CNC 0), 0.15% w/v (PL-CNC 0.15), 0.31% w/v (PL-CNC 0.31), 0.45% w/v (PL-CNC 0.45), and 0.61% w/v (PL-CNC 0.61) in 50% PL composition.

FIG. 5: In vitro evaluation of cell supportive properties. Live/Dead staining with Calcein AM/PI (green: live cell; red: dead cell) of hASCs encapsulated in PL/CNC hydrogels (A).

FIG. 6: Hydrogels retraction upon hASCs encapsulation and analysis of adhesion and morphology of hASCs encapsulated. Photographs of PL-CNC hydrogels after 3 hours and 7 days in culture (A). Hydrogels retraction in percentage at 1, 4 and 7 days (B). Fluorescence microscopy images showing cytoskeleton organization in the fibrin matrix after 1 day in culture (C). Fluorescence microscopy images showing cytoskeleton organization after 1 and 3 days in culture (D). Cell axial ratio and cell spreading area after 1 day of culture were quantified for all conditions (E) and cell axial ratio and cell spreading area after 1 and 3 day of culture were quantified for PL-CNC 0.61 (F). Staining fibrinogen (green), actin (red) and nuclei (blue). *P<0.05 PL-CNC 0 vs PL-CNC (0.15-0.46). +P<0.05 PL-CNC 0.31 vs PL-CNC (0.61). & P<0.05 PL-CNC 0.46 vs PL-CNC 0.61. #P<0.05 between 1 and 3 days in culture. Scale bars: 4 mm (A); 10 μm (C); 50 μm (D).

FIG. 7: hASCs were assessed for the expression of chondrogenic (Sox-9 and COMP), osteogenic (Runx2, Cola1 and ALP), adipogenic (LPL) and angiogenic markers (PDGF and VEGF) on PL-CNC 0, 0.31 and 0.61 hydrogels (A). *P<0.05 PL-CNC 0 vs PL-CNC (0.31-0.46). #P<0.05 PL-CNC 0.31 vs PL-CNC 0. /P<0.05 PL-CNC 0.61 vs PL-CNC (0-0.31). & P<0.05 between 1 and 7 days in culture.

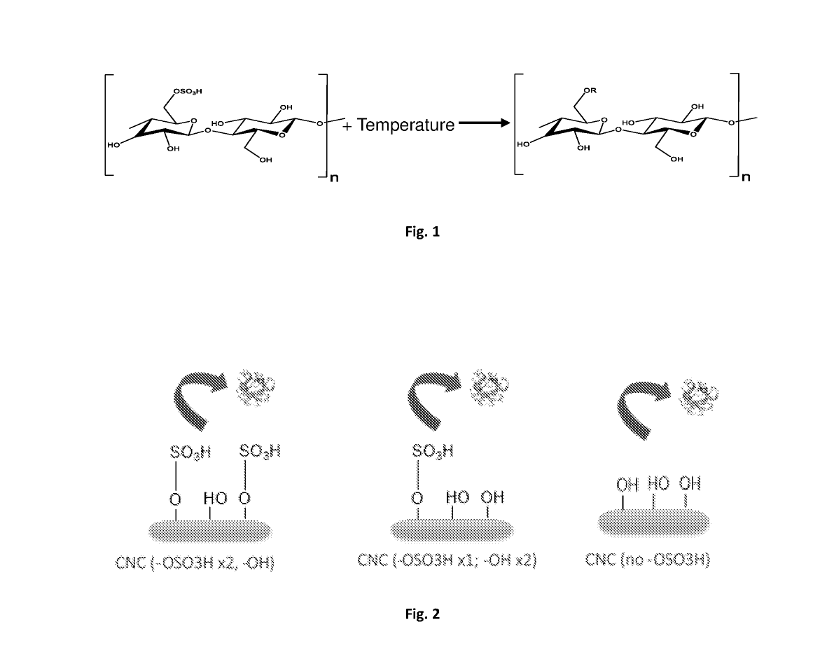

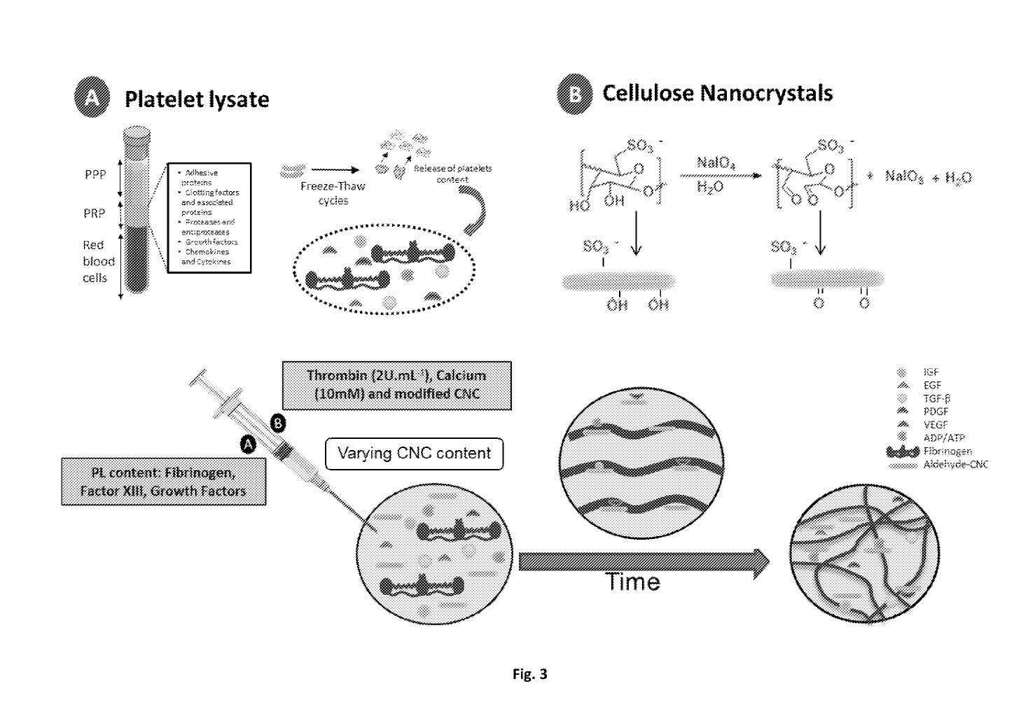

### DETAILED DESCRIPTION

The present disclosure comprises the use of CNC as multifunctional nanofillers in BD-based material that can act as 1) reinforcing nanofillers and crosslinkers of the protein matrix and as 2) sulfated glycosaminoglycan mimetic entities to reversibly sequester platelet-derived GFs and/or other soluble biomolecules in a three-dimensional (3D) microenvironment.

In an embodiment, the present disclosure enables the use of BD not only as growth factors cocktail but also as scaffolding biomaterial by crosslinking its protein content with oxidized CNC.

In an embodiment, platelet concentrates (PC) were obtained from different platelet collections produced by plasmapheresis. The platelet count was performed using the COULTER® LH 750 Hematology Analyzer and the sample volume adjusted to 1 million platelet μL⁻¹.

In an embodiment, PC batches were subjected to three repeated temperature cycles (frozen with liquid nitrogen at −196° C. and melt in a 37° C. water bath), lysing the platelets and releasing their protein content. The lysate was then centrifuged at 4000 G for 5 min at 5° C. and filtered through a 0.45 μm pore filter to reduce platelet membrane fragments. Aliquots of platelet lysate (PL) were stored at −80° C. until final use.

In an embodiment, CNC can be extracted from microcrystalline cellulose (MCC) powder, in particular from cotton, wood, or other suitable sources (following the typical sulfuric acid hydrolysis).

In an embodiment, 42 g of MCC were suspended in 189 ml of deionized water (DI) cooled in an ice bath using a mechanical agitator (500 rpm) during 10 minutes. Concentrated sulfuric acid (95%-97%, 188.3 mL) was added dropwise up to a final concentration of 64 wt % under mechanical stirring. The reaction was performed under continuous stirring at 44° C. for 120 min, stopped by addition of 5 fold excess cold water and left to decant at 4° C. The supernatant was discarded and the remaining suspension was centrifuged three times for 10 min at 9000 rpm and 5° C. The supernatant was successively replaced with DI water and the suspension subjected to centrifugation cycles until the supernatant became turbid. The resulting suspension was collected and extensively dialyzed against DI water until neutral pH. After dialysis the content was sonicated for 10 min using an ultrasound probe at 60% of amplitude output, under ice cooling to prevent overheating. The cloudy suspension was centrifuged one last time to remove big particulates and the final supernatant containing the CNC was stored at 4° C. until further use.

In an embodiment, oxidation was performed to convert CNC surface hydroxyls to carbonyls. The carbonyls are expected to induce covalent crosslinking between CNC and platelet-derived proteins.

In an embodiment, aldehyde functionalized CNC were produced by sodium periodate oxidation. In a preferred embodiment, sodium periodate is added to CNC aqueous suspension (1.5 wt %) in a 1:1 molar ratio (sodium periodate/anhydroglucose equivalents). The mixture is allowed to stir at room temperature for 12 hours preventing from light exposure. Unreacted periodate was quenched by the addition of ethylene glycol. The mixture is transferred into a dialysis membrane and dialyzed against ultrapure water for 3 days with regular water replacement. The final suspension is then collect and stored at 4° C. until further use.

In an embodiment, CNC suspensions were submitted to a hydrothermal treatment process to reduce the surface sulfate content of the initial condition, FIG. 1. An aqueous suspension of CNC (1 wt %) was added to a autoclave. The autoclave was sealed and heated to 120° C., and held at the desired temperature for different time periods (4 h to 20 h) in order to obtain different sulfation gradients. After cooling the autoclave to room temperature, CNC suspension was collected and stored at room temperature in sealed glass vials until further characterization was performed.

In an embodiment, CNC suspension was further characterized by conductometric titration, based on Beck and co-workers method [1], where a certain sulfation degree is obtain 100 mmolKg-1 to 300 mmolKg-1 (mmol sulfate groups per 1 kg of cellulose).

In an embodiment, conductometric titration was determined. The carbonyl group content of the oxidized aldehyde CNC (a-CNCs) was determined by conductometric titration according to [2]. In a typical run, 3.6 mL of a-CNC aqueous suspension (1.39 wt. %, 0.050 g) and 0.025 g (0.62 mmol) of NaOH were dispersed in a final volume of 10 mL of ultra-pure water. 0.193 g of silver (I) oxide were added to the solution which was allowed to stir overnight and selectively oxidize the aldehyde groups to carboxylic acids. 5 mL of the oxidized reaction mixture were diluted with 80 mL of ultra-pure water and the pH was adjusted to c.a. 3.5 with HCl 1M. Finally, the solution was titrated using 0.01M NaOH. The total amount of carboxyl groups corresponding to the carbonyl content or degree of oxidation (DO) was calculated from:

$$D O = \frac{162 C ( {V}_{2} - {V}_{1} )}{w - 36 C ( {V}_{2} - {V}_{1} )} \qquad ( Equation S1 )$$

where C is the NaOH concentration (mol/L), V1 and V2 are the amount of NaOH, and w (g) is the weight of a-CNC.

In an embodiment, CNC dimensions were analysed by Atomic Force Microscopy (AFM). CNC produced were analysed by AFM to determine the particles size distribution. Drops of the diluted CNC suspension (0.0015 wt. %) were deposited on freshly cleaved and carefully washed mica discs (9.9 mm diam. 0.27 thick). The suspension was left to adsorb for 15 minutes and the excess liquid was removed. The disc was allowed to dry overnight. The samples ware imaged in tapping mode with a MultiMode AFM connected to a NanoScope V controller, both from Veeco, USA, with non-contact silicon nanoprobes (c.a. 300 kHz) from Nanosensors (Switzerland). The particle size distribution was determined with Gwyddion software.

### Nanocomposite Formulations

In an embodiment, nanocomposite formulation of the present disclosure, physiologically stable and mechanically reinforced BD loaded in CNC nanocomposites, can be produced by combining oxidized CNC suspensions with a different degree of sulfation with BD formulations.

In an embodiment, the aldehyde groups of oxidized CNC reversible react with amine groups of platelet-derived proteins through Schiff's base reaction and crosslink the protein matrix.

In a particular embodiment, thrombin and calcium may be used to maximize the crosslinking of platelet-derived proteins (fibrinogen) and to allow for the production of injectable nanocomposite materials that can crosslink in situ at physiological conditions.

TABLE I
Components concentration in the final formulation

| | | | | | Sulfation |
|---|---|---|---|---|---|
| | | | | BD | degree |
| | a-CNC | Thrombin | Calcium | platelets/ | of a-CNC |
| Values | % w/v | U.mL | mM | μL | mmol · Kg⁻¹ |
| Minimum | 0.05 | 0.1 | 0.1 | 0.5 × 10⁴ | 50 |
| Studied | 0.15-0.61 | 1 | 5 | 11 × 10⁶ | 100-300 |
| Maximum | 2 | 50 | 25 | 2 × 10⁸ | 500 |

### EXAMPLE 1

In an embodiment, sponges were prepared at room temperature using a double-barrel syringe fitted with a static mixer to ensure an effective mixing of the nanocomposite components.

Barrel A was filled with PL and barrel B with oxidized CNC presenting a certain sulfation degree (100 mmolKg⁻¹ to 300 mmolKg⁻¹).

Aqueous suspensions of CNC with varying concentrationsof 0% w/v (PL-CNC 0), 0.15% w/v (PL-CNC 0.15), 0.31% w/v (PL-CNC 0.31), 0.45% w/v (PL-CNC 0.45), and 0.61% w/v (PL-CNC 0.61) in 50% PL composition.

The PL/CNC mixtures were frozen and freeze-dried to produce PL/CNC nanocomposite sponges.

PL/CNC nanocomposite sponges were prepared in cylindrical acrylic molds of 9 mm diameter and 5 mm height. Alternatively, the PL/CNC mixtures may be poured into any form or mold having the desired final material shape.

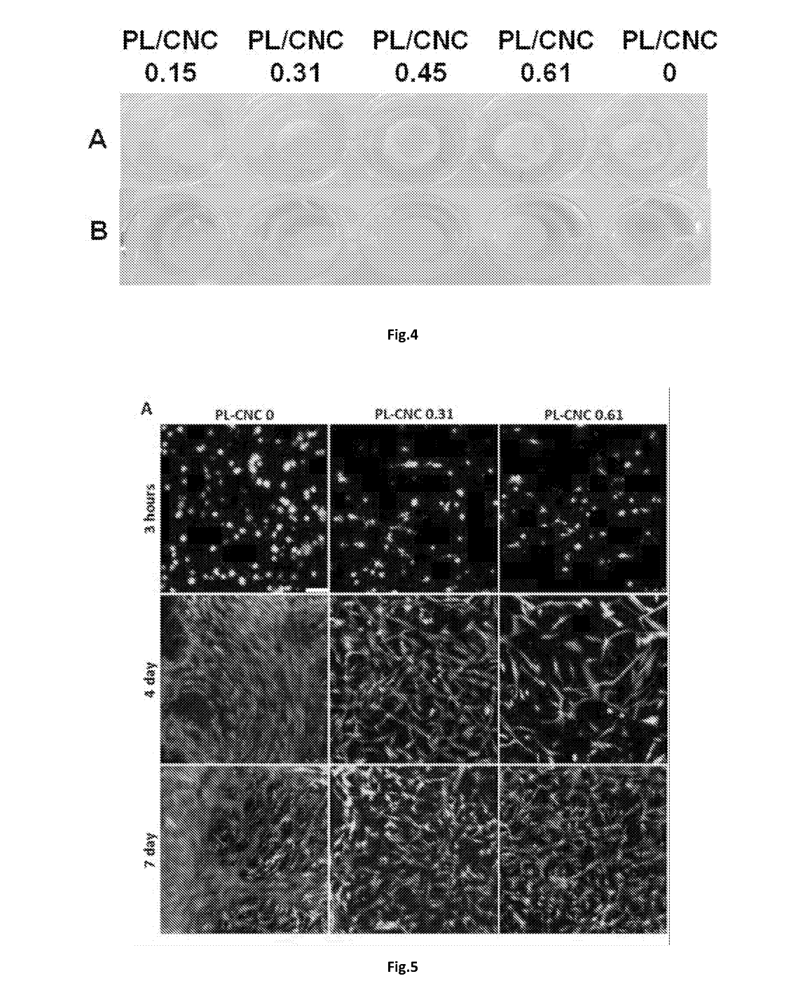

In an embodiment, CNC incorporation lead to an improvement of PL stability (FIG. 4) and hydrogels with higher CNC content showed lower degradation rate.

In an embodiment, CNC incorporation leads to a more organized microstructure with smaller pores.

In an embodiment, the porosity (vol. %) increases from 64.6 to 75.1 with increasing of CNC content. Increasing CNC content significantly improved the mechanical properties (compression modulus and strength) of PL/CNC spongy hydrogels.

Fast recover of initial shape upon unloading demonstrate the high elastic nature of PL/CNC spongy hydrogels.

PL/CNC conditions demonstrated cellular viability after 9 days in culture.

### EXAMPLE 2

In an embodiment, sponges were prepared at room temperature using a double-barrel syringe fitted with a static mixer to ensure an effective mixing of the nanocomposite components.

Barrel A was filled with PL and barrel B with oxidized CNC presenting a certain sulfation degree (100 mmolKg⁻¹ to 300 mmolKg⁻¹), calcium, and thrombin.

Aqueous suspensions of CNC with varying concentrations of 0% w/v (PL-CNC 0), 0.15% w/v (PL-CNC 0.15), 0.31% w/v (PL-CNC 0.31), 0.45% w/v (PL-CNC 0.45), and 0.61% w/v (PL-CNC 0.61) in 50% PL composition.

The precursor solutions were then hand extruded into cylindrical acrylic molds of 9 mm diameter and 5 mm height and incubated at 37° C. for a certain period of time to allow fibrin fibrillation to proceed. Alternatively, the PL/CNC mixtures may be poured into any form or mold having the desired final material shape.

The PL/CNC mixtures were frozen and freeze-dried to produce crosslinked PL/CNC nanocomposite sponges.

### EXAMPLE 3

In an embodiment, hydrogels were prepared at room temperature using a Double-barrel syringe (1:1) with a mixer tip was used to produce this system (L-System, Medmix, Switzerland). promoting the in situ PL-clotting via thrombin and calcium activation along with the CNC/protein covalent crosslinking.

Barrel A was filled with PL (67.6 mg/mL of total protein) composed of albumin, growth factors, cytokines and structural proteins (such as fibrinogen, vitronectin and fibronectin) [3, 4].

Barrel B was composed of thrombin (2 U.mL-1), calcium (10 mM) and a-CNC water dispersions presenting a certain sulfation degree (100 mmolKg⁻¹ to 300 mmolKg⁻¹).

Aqueous suspensions of CNC with varying concentrations of 0% w/v (PL-CNC 0), 0.15% w/v (PL-CNC 0.15), 0.31% w/v (PL-CNC 0.31), 0.45% w/v (PL-CNC 0.45), and 0.61% w/v (PL-CNC 0.61) in 50% PL composition.

The precursor solutions were then hand extruded into cylindrical acrylic molds of 9 mm diameter and 5 mm height and incubated at 37° C. for a certain period of time to allow fibrin fibrillation to proceed. Alternatively, the PL/CNC mixtures may be poured into any form or mold having the desired final material shape or be injectable extruded in the tissue injury defect.

In an embodiment, the gelation, microstructural, mechanical, swelling, degradation and protein release profiles of the hydrogels were fully characterized.

In optimized conditions for PL gelation (1 U.mL⁻¹ thrombin and 5 mM CaCl₂), incorporation of up to 0.61% w/v CNC considerably improved the microstructural organization, stability and degradation rate of the hydrogels. Moreover, the proposed strategy did not hinder a fast gelation process while markedly increased the hydrogels mechanical properties up to an impressive 2 orders of magnitude higher storage modulus compared to control (maximum G′ of 1.2 kPa) and improved their ability to sequester native PL bioactive factors.

In an embodiment, at nanoscale dimensions using AFM measurements, CNC incorporation leads to higher young's modulus in PL-CNC hydrogels. In PL-CNC 0 formulation was 2.2 kPa being very similar of the values obtained along the fibers (1.3 kPa). The increased bundle thickness and sample heterogeneity in PL-CNC 0.46-0.61 was correlated with higher fiber rigidity. Differences between gel and fiber rigidity are higher when higher CNC concentration are obtained, which indicates the incorporation of CNC along the fibers. Cell-scale measurements, as well as, bulk rheological properties, showed increased stiffness for higher CNC loading. As expected, mechanical properties can be modulate by tailoring CNC concentration and network densities.

In an embodiment, PL-CNC 0 hydrogels rapidly degraded over this time in comparison of PL-CNC (0.15-0.61) formulations. To examine the ability to sequester native PL bioactive factors from degradation within PL-CNC hydrogels, total protein released was quantified. The hydrogels were incubated in PBS and each day fresh PBS was replaced. After 6 days in PBS, PL-CNC 0 matrix was almost completely degraded. In contrast, in PL-CNC 0.61 around 61% of the protein hydrogel is maintained over 7 days. Altogether these data indicate that different matrix arquitectures and composition can influence the interactions and released of PL bioactive molecules from within the hydrogel.

In an embodiment, concerning proliferative capacities of the PL-CNC hydrogels, it is visible that improving structure stability leads to higher number of cells encapsulated over time (FIG. 5). After 7 days in culture, metabolic activity normalized with DNA content showed that CNC incorporation improves cell activity.

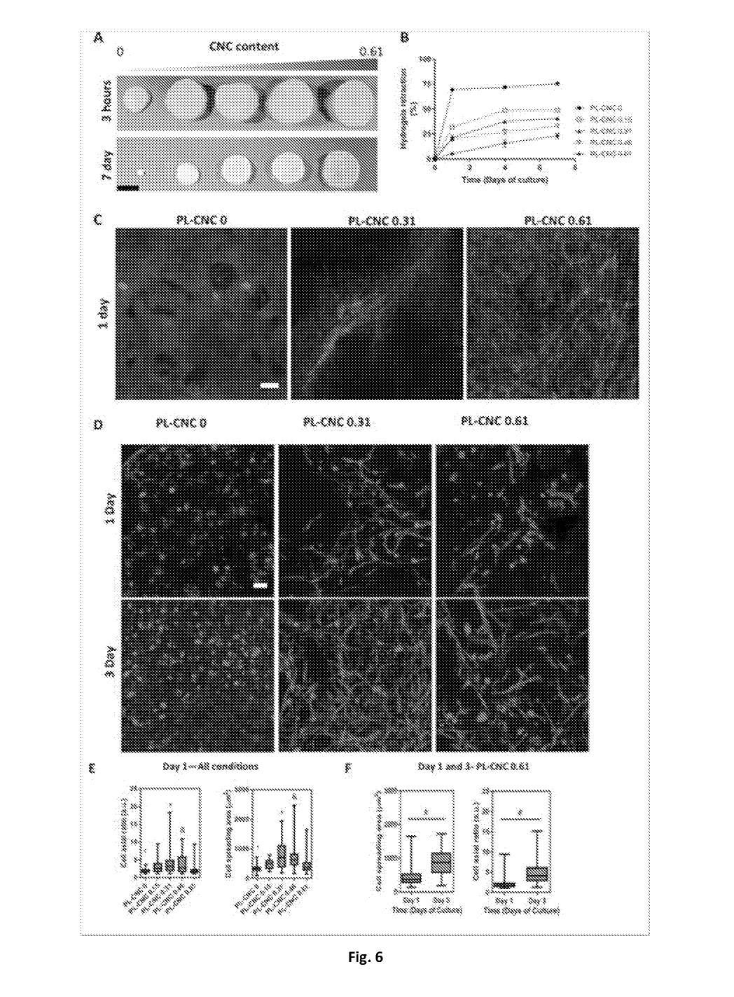

In an embodiment, upon hASCs encapsulation, PL hydrogel has a manifold densification of the fibrin network referred to as clot retraction. PL-CNC 0 exert a modest contractile effect that results in 75% reduction in total diameter (FIG. 6).

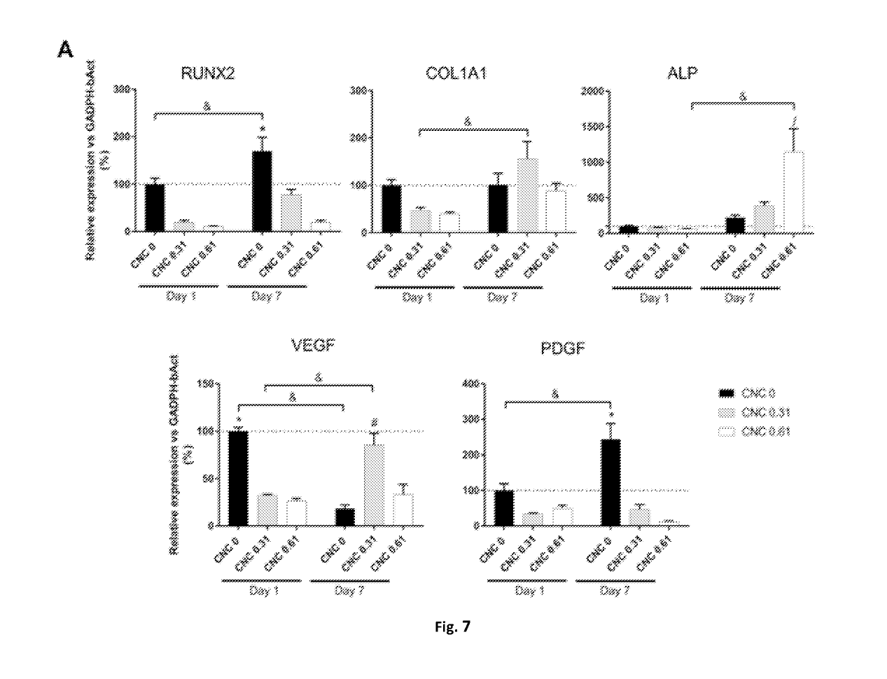

In an embodiment, concerning chondrogenic differentiation, it was studied sex-determining region Y-box 9 protein (SOX-9), as a key transcription factor in early chondrogenesis and cartilage oligomeric matrix protein (COMP), which is one of the major matrix molecules in articular cartilage (FIG. 7). In order to study osteogenic potential, human adipose derived-stem cells (hASCs) were examined for the expression of gene expression during early phases, runt-related transcription factor 2 (RUNX2), collagen Type I Alpha 1 Chain (COL1A1) and alkaline phosphatase (ALP). Specifically, RUNX2 is crucial for the generation of a mineralized tissue, COL1A1 is the main constituent of the bone organic part of the extracellular matrix (ECM) and ALP is responsible for the mineralization of the ECM [5]. In vitro osteogenic differentiation can also be predicted by the ratio between RUNX2 and SOX-9, since SOX9 directly interacts with Runx2 and represses its activity [6]. The increased expression of osteogenic markers (ALP and COL1A1), downregulation of chondrogenic-related markers (SOX9 and COMP) RUNX2/SOX-9 ratios showed significant higher values on Day 7 compared on Day 1 in PL-CNC incorporated CNC suggested that presence of CNC tend to differentiate hASCs in osteogenic lineage which is in agreement within the established paradigm of stiffness-directed stem cells differentiation [7].

In an embodiment, the disclosure platform allows therefore using PL as stable injectable formulations for either the delivery of biological factors as well as a cell carrier matrix. Overall, this platform open new avenues to explore PL based hydrogels in TE applications, enabling a controlled modulation of the physical and chemical cellular microenvironments in in vitro settings, as well as upon in vivo injection. Their 3D in vitro biological performance was assessed using encapsulated hASCs. Hydrogels formulations showed cell supportive properties, such as viability, metabolic activity, and proliferation rate.

In an embodiment, the materials of the present disclosure relies on the production of structures composed of PL reinforced with varying contents of aldehyde-modified CNC and a certain sulfation degree. Aldehyde-modified CNC baring surface aldehyde groups reversibly react with terminal amine groups of proteins, which improve the spongy hydrogel structural integrity and mechanical properties. The present disclosure discloses the feasibility of incorporating modified CNCs into PL-based scaffolds and shown its structural and biological performance.

In an embodiment, the hydrogel produced using this method may be used as an injectable biomaterial able to crosslink in physiological conditions.

In an embodiment, the injectable PL/CNC may be applied as a cell carrier or as an acellular material in medical applications.

The term “comprising” whenever used in this document is intended to indicate the presence of stated features, integers, steps, components, but not to preclude the presence or addition of one or more other features, integers, steps, components or groups thereof.

Where singular forms of elements or features are used in the specification of the claims, the plural form is also included, and vice versa, if not specifically excluded. For example, the term “a cell” or “the cell” also includes the plural forms “cells” or “the cells,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

The following references should be considered here with incorporated in their entirety:
1. Beck, S., M. Methot, and J. Bouchard, General procedure for determining cellulose nanocrystal sulfate half-ester content by conductometric titration. Cellulose, 2015. 22(1): p. 101-116.
2. Domingues, R. M., et al., Development of injectable hyaluronic acid/cellulose nanocrystals bionanocomposite hydrogels for tissue engineering applications. Bioconjugate chemistry, 2015. 26(8): p. 1571-1581.
3. Fekete, N., et al., Platelet lysate from whole blood-derived pooled platelet concentrates and apheresis-derived platelet concentrates for the isolation and expansion of human bone marrow mesenchymal stromal cells: production process, content and identification of active components. Cytotherapy, 2012. 14(5): p. 540-554.
4. Crespo-Diaz, R., et al., Platelet lysate consisting of a natural repair proteome supports human mesenchymal stem cell proliferation and chromosomal stability. Cell transplantation, 2011. 20(6): p. 797-811.
5. Granéli, C., et al., Novel markers of osteogenic and adipogenic differentiation of human bone marrow stromal cells identified using a quantitative proteomics approach. Stem cell research, 2014. 12(1): p. 153-165.
6. Loebel, C., et al., In vitro osteogenic potential of human mesenchymal stem cells is predicted by Runx2/Sox9 ratio. Tissue Engineering Part A, 2014. 21(1-2): p. 115-123.
7. Discher, D. E., D. J. Mooney, and P. W. Zandstra, Growth factors, matrices, and forces combine and control stem cells. Science, 2009. 324(5935): p. 1673-1677.
8. WO2014077854A1.
9. WO2013116791A1.

1. A composition comprising:
a blood derivative component;
a cellulose nanocrystal as a filler comprising carbonyls at the cellulose nanocrystal surface,
wherein such cellulose nanocrystal comprises a sulfation degree of at least 50 mmolKg-1.

2. The composition according to claim 1 wherein the sulfation degree is between 80 and 500 mmolKg-1.

3. The composition according to claim 1 wherein the blood derivative component is platelet-rich plasma, a platelet cellular component, a platelet lysate or a platelet released content.

4. The composition according to claim 1 comprising 0.05-2% w/v of cellulose nanocrystal.

5. The composition according to claim 1 comprising 0.5×104 platelets/μL-2×108 platelets/μL of blood derivative platelet concentration.

6. The composition according to claim 1 further comprising thrombin, calcium, calcium salts, or mixtures thereof.

7. The composition according to claim 6 comprising 0.1 U.mL-1-50 U.mL-1 of thrombin.

8. The composition according to claim 1 comprising 0.1 mM-25 mM of calcium or a calcium derivate.

9. The composition according to claim 1 comprising an amount of carbonyl groups at the surface of the cellulose nanocrystals between 0.01-8 mmol.g-1.

10. The composition according to claim 1 further comprising one or more active ingredients or biomolecules.

11. The composition according to claim 10 wherein such active ingredient or biomolecule is selected from the group consisting of: a drug; an active ingredient, a growth hormone, a cell attractant, a drug molecule, a cell, a bioactive glass, a tissue growth promoter, and combinations thereof.

12. The composition according to claim 11 wherein the drug molecule is an anti-inflammatory, antipyretic, analgesic, anticancer agent, or mixtures thereof.

13. The composition according to claim 11 wherein cells are selected from the group consisting of: osteoblasts, osteoclasts, osteocytes, pericytes, endothelial cells, endothelial progenitor cells, bone progenitor cells, hematopoietic progenitor cells, hematopoietic stem cells, neural progenitor cells, neural stem cells, mesenchymal stromal/stem cells, induced pluripotent stem cells, embryonic stem cells, and combinations thereof.

14. The composition according to claim 1 further comprising one or more pharmaceutically acceptable excipients.

15. The composition according to claim 14 wherein such pharmaceutically acceptable excipient is selected from the group consisting of: an additive, a binder, a disintegrant, a diluent, a lubricant, a plasticizer, and mixtures thereof.

16. The composition according to claim 1 wherein the average length of the cellulose nanocrystal is between 40-2500 nm.

17. The composition according to claim 1 wherein the average width of the cellulose nanocrystal is between 3-50 nm.

18. The composition according to claim 1 wherein the blood derivative component is obtainable by centrifugation or by apheresis.

19. A pharmaceutical or cosmetic composition comprising the composition of claim 1.

20. (canceled)

21. A method for treating a wound or healing a tissue injury defect in a subject comprising administering the compound of claim 1 to the subject requiring treatment or therapy of wound healing or a tissue injury defect.

22. The method of claim 21 for the treatment or therapy of defects of skin wound, orthopedic injury, pain, nerve disease, dental injury, bone injury, or diabetic wound healing.

23. The pharmaceutical composition according to claim 19, wherein the pharmaceutical composition is an injectable formulation.

24. A hydrogel comprising the composition of claim 1 comprising a blood derivative component, oxidized cellulose nanocrystals of a certain sulfation degree, thrombin and/or calcium through chemical crosslinking.

25. The hydrogel according to claim 24 wherein the cells are encapsulated or seeded.

26. The hydrogel according to claim 25 wherein the hydrogel is an in situ crosslinked injectable hydrogel at physiological conditions.

27. The hydrogel according to claim 24 wherein the hydrogel is casted to the desired mold shape or be in situ injectable extruded.

28. A sponge or scaffold comprising the composition of claim 1 comprising a blood derivative component reinforced with oxidized cellulose nanocrystals of a certain sulfation degree.

29. The sponge or scaffold according to claim 28 further comprising a cell, oxidize cellulose nanocrystals, thrombin and/or calcium through chemical crosslinking.

30. The sponge or scaffold according to claim 24 wherein the sponge is casted to the desired mold shape.

31. The sponge or scaffold according to claim 24 wherein the cells are encapsulated or seeded.

32. A method for the preparation of the composition of claim 1, comprising the following steps:
obtaining oxidized cellulose nanocrystals comprising a sulfation degree of at least 50 mmolKg-1, by a hydrothermal treatment of the oxidized cellulose nanocrystal; and
mixing a blood derivative component with the oxidized cellulose nanocrystal.

33. The method according to claim 32 further comprising the addition of calcium and/or thrombin to the mixture.

34. The method according to claim 32 wherein the blood derivative component is platelet-rich plasma, a platelet cellular component, a platelet lysate and/or a platelet released content.
